(19) United States
(12) Patent Application Publication
(10) Pub. No.: US 2022/0138949 A1
(43) Pub. Date: May 5, 2022

(54) System and Method for Tissue Classification Using Quantitative Image Analysis of Serial Scans

(71) Applicant: The Regents of the University of California, Oakland, CA (US)

(72) Inventors: Dieter Enzmann, Los Angeles, CA (US); William Hsu, Oakland, CA (US); Corey Arnold, Oakland, CA (US)

(21) Appl. No.: 17/431,353

(22) PCT Filed: Feb. 20, 2020
(86) PCT No.: PCT/US2020/019076

§ 371 Date: Aug. 16, 2021

Related U.S. Application Data
(60) Provisional application No. 62/807,811, filed on Feb. 20, 2019.

(51) Int. Cl. G06T 7/00; G06T 5/00; G06T 7/11; G16H 30/40

(52) U.S. Cl. CPC G06T 7/0016; G06T 5/002; G06T 7/11; A61B 6/502; G06T 2207/20076; G06T 2207/20084; G16H 30/40

(57) ABSTRACT

A method for tissue classification includes receiving at least two images associated with a patient, the at least two images being of an anatomical region or tissue. The method also includes identifying a region of interest in the at least two images, analyzing the region of interest to identify changes in the tissue and generating a probability map of the region of interest based on the changes in the tissue. The probability map indicates a likelihood of formation of cancer in the tissue within a predetermined time period. The method also includes displaying the probability map on a display.

### CROSS-REFERENCE TO RELATED APPLICATION

This application is based on, claims priority to, and incorporates herein by reference in its entirety U.S. Ser. No. 62/807,811 filed Feb. 20, 2019 and entitled “System and Method For Tissue Classification Using Quantitative Image Analysis of Serial Scans.”

### BACKGROUND

Advances in biotechnology have generated large quantities of detailed information on patients' genetic risk for cancer such as the presence of cancer-promoting germline mutations and highly sensitive blood tests that can detect tumor DNA circulating in the blood. While these developments have the potential to improve the detection of cancer at earlier stages at the microscopic level, medical imaging remains an integral player in localizing and characterizing observable changes at the macroscopic level. The current imaging paradigm for cancer screening and detection to visually search for any signs of cancer. While screening for cancer (e.g., mammography for breast cancer) has been shown to reduce cancer related mortality, interpretation of screening exams is imperfect. For breast imaging, for example, nationally one in eight cancers have been found to go undetected by radiologists and 10 percent of all screening exams are called back for diagnostic workup, with a majority being false-positive results. Improving the interpretation of screening images, for example, mammograms, would minimize potential harms and enhance benefits to the population of being screened. In recent years, machine learning, including deep learning, has increasingly been utilized in the analysis of medical images (e.g., image recognition, disease classification) as a result of increased computational power and accumulation of big data.

It would be desirable to provide a system and method for image-based tissue classification that utilize advanced machine learning approaches combined with information from advanced biotechnology to predict the formation of cancer (e.g., determine a probability of cancer formation) before a distinct abnormality is observed. A determination of a prediction or probability of cancer formation for a given patient may be valuable for treatment planning and decision making regarding treatment.

### SUMMARY OF THE DISCLOSURE

In accordance with an embodiment, a method for tissue classification includes receiving at least two images associated with a patient, the at least two images being of a tissue, identifying a region of interest in the at least two images, analyzing the region of interest to identify changes in the tissue, generating a probability map of the region of interest based on the changes in the tissue, the probability map indicating a likelihood of formation of cancer in the tissue within a predetermined time period and displaying the probability map on a display.

In accordance with another embodiment, a system for tissue classification includes at least one database and a preprocessing module. The preprocessing module is coupled to the at least one database and configured to receive at least two images associated with a patient, the at least two images being of a tissue, to identify a region of interest in the at least two images, and to analyze the region of interest to identify changes in the tissue. The system also includes a classifier coupled to the at least one database and the preprocessing module and configured to generate a probability map of the region of interest based on the changes in the tissue, the probability map indicating a likelihood of formation of cancer in the tissue within a predetermine time period.

### BRIEF DESCRIPTION OF THE DRAWINGS

FIG. 1A is a block diagram of a system for tissue classification in accordance with an embodiment;

FIG. 1B is a block diagram of a system for training a classifier of the system shown in FIG. 1A in accordance with an embodiment;

FIG. 2 illustrates a method for training a classifier in accordance with an embodiment;

FIG. 3A illustrates a method for generating difference image data for training a classifier in accordance with an embodiment;

FIG. 3B illustrates a method for generating reference data for training a classifier in accordance with an embodiment;

FIG. 4 illustrates a method for tissue classification in accordance with an embodiment; and

FIG. 5 is a block diagram of an example computer system that can implement the systems and methods described herein in accordance with an embodiment.

### DETAILED DESCRIPTION

The present disclosure describes a system and method for image-based tissue classification and quantitative prediction. The computer-vision based system and method is configured to analyze medical images (e.g., images obtained for detecting the presence of cancer or diagnostic images) in the context of known germline mutations that have been identified using molecular or genomic analysis such as whole genome sequencing (WGS) or whole exome sequencing (WES). The system and method may be used to analyze tissue in an organ to assess its favorability status for initiating forming or growing a cancer given a known genetic risk (e.g., alterations to specific genes such as BRCA1/2). For example, a quantitative prediction of the probability of the formation of cancer in a tissue may be generated based on an analysis of images of the tissue. It has been shown that multiple variegated biologic networks display abnormal behavior or dynamics before a cancer forms, and these abnormalities change microenvironmental tissue states for a cancer cell to survive, “take root,” and progress. Image analysis of microenvironmental tissue states may reflect the presence of abnormal biologic networks, i.e., cellular, tissue organ, and systemic network abnormalities and thus detect the early pre-cancer cell environment. The system and method described herein is configured to identify “pre-cancer” tissue status rather than to search for an already formed cancer as is done in current imaging paradigms for detecting cancer. In various embodiments, the system and method described herein is configured to identify static and dynamic tissue imaging characteristics that indicate a “fertile ground” for cancer development in the presence of known elevated genetic risks.

It may be advantageous for an individual with a known cancer-promoting germline mutation to assess the personal risk of cancer developing in certain tissues or organs. For example, a germline mutation such as BRCA1/2 increases the risk of breast cancer while also increasing the risk of cancer in other tissues (e.g., fallopian tube/ovarian). The system and method described herein may be valuable for clinical decision-making on the part of patients who have a baseline risk as determined by WGS and WES analysis (or some other technology) and complemented by relevant phenotype changes in the tissues at risk. For example, knowing a probability of cancer formation provided by the system and method for tissue classification may be valuable to patients considering drug treatment, such as aromatase inhibitors, to prevent or delay possible cancer formation. Given a genetic predisposition for cancer development, the goal of computationally analyzing tissue with no obvious abnormalities is to detect subtle changes that reflect early time-sequenced biologic network perturbations that may eventually lead to actual cancer formation, which requires multiple sequential and parallel tissue factors to develop for a cancer to start, survive and grow. Monitoring of unobvious changes in tissue may be referred to as the “countdown” to cancer.

To detect these tissue changes, a computer-based system may be used that incorporates machine learning (ML) and deep learning (DL) methods to detect subtle static and dynamic incremental tissue (or whole organ) imaging features difficult to detect by the human visual system. Machine and deep learning may be used to extract information from medical images. In an embodiment, the longitudinal nature of the data collected in images (e.g., detection or diagnostic images) may enable utilization of advanced machine learning approaches to analyze subtle changes in the appearance of an imaged region (e.g., breast parenchyma) that may predict the formation of cancer even before a distinct abnormality is observed. In an embodiment, an individualized probability map that visualizes the risk of observed changes in pixel or voxel values as reflecting malignancy within a specific time period may be generated and presented for a given patient. The probability map may be valuable to, for example, radiologists and referring physicians, in determining how best to move forward with further diagnostic tests, particularly in patients with an observed mutation (e.g., BRCA1 positive). The probability map may be useful for shared decision making.

In an embodiment, the system and method for tissue classification may be used to characterize tissue changes that presage formation of actual cancer. In another embodiment, the system and method for tissue classification may also be used to identify cancer. Salient imaging features (e.g., intensity profile, shape, texture) may be quantitatively analyzed and associated with changes in tissue characteristics (e.g., microstructure, metabolic status, physiologic status, cytoarchitecture, etc.) that indicate a pathway favorable to cancer formation. The system and method for tissue classification may be used to provide quantitative predictions for the formation of various types of cancer such as, for example, breast cancer, prostate cancer, liver cancer, pancreas cancer, etc.

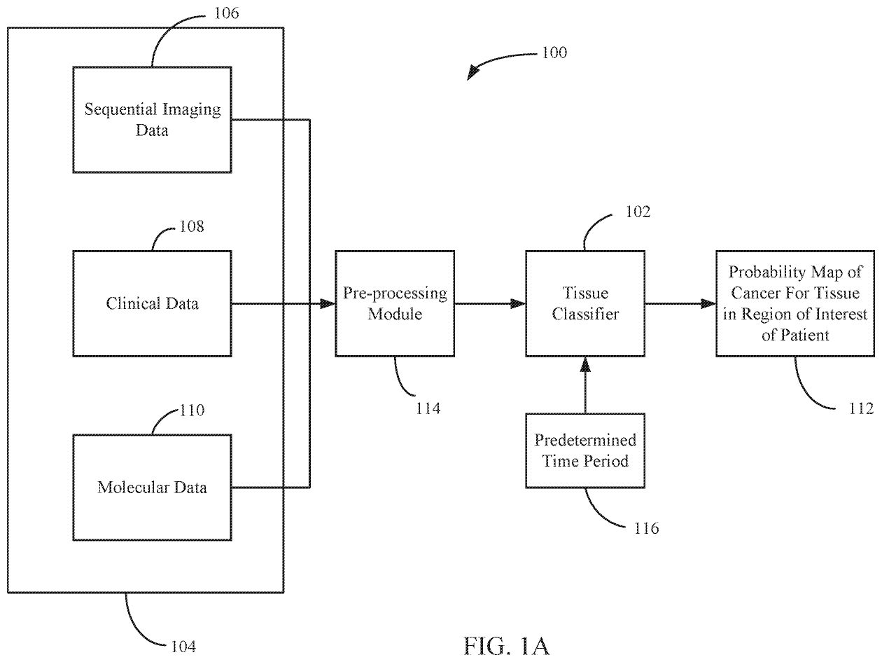

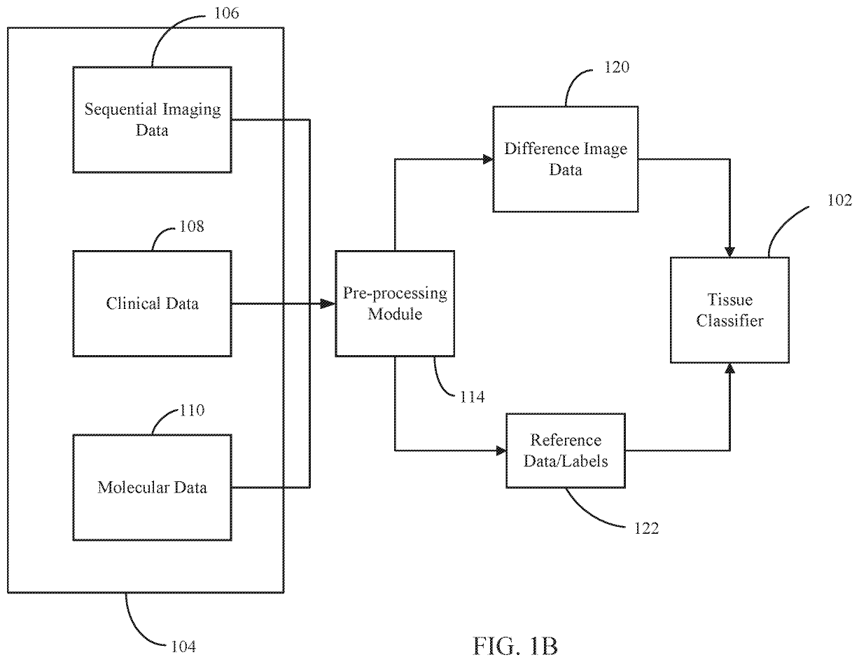

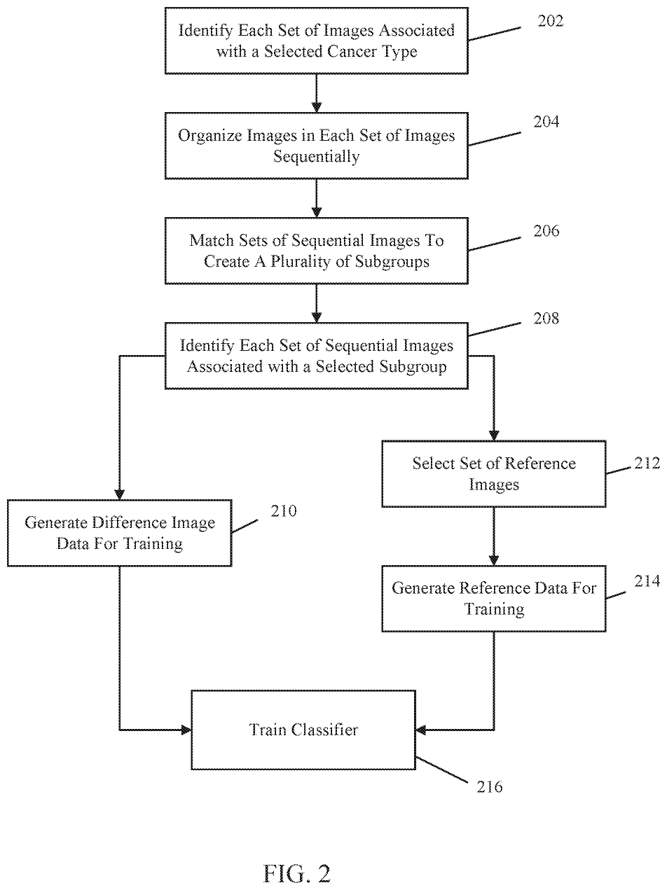

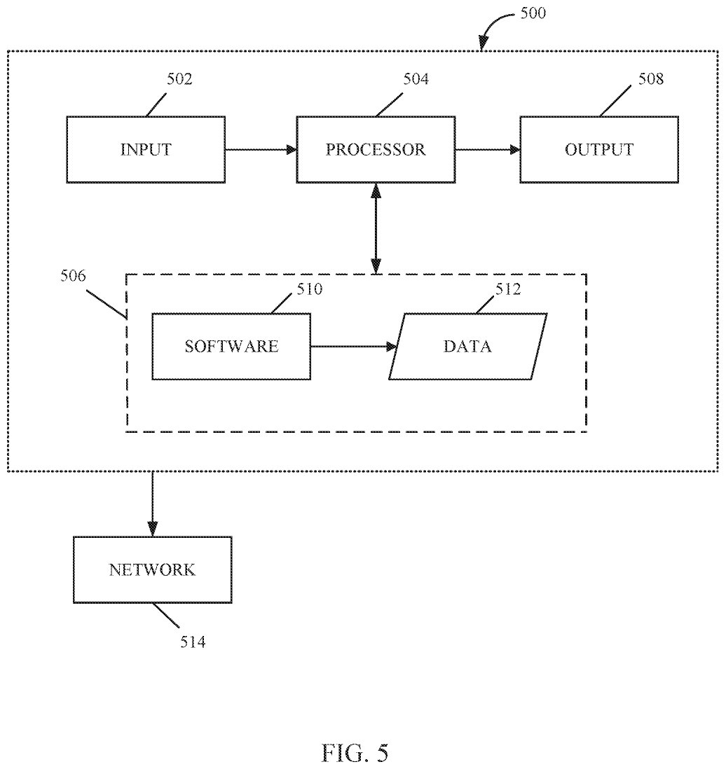

FIG. 1A is a block diagram of a system for tissue classification in accordance with an embodiment. The system 100 includes a classifier 102 and a pre-processing module 114 that are coupled to one or more databases 104. The classifier 102 is configured to receive data as input from the one or more database 104 that may include imaging data 106 (e.g., sequential imaging data), clinical data 108 and molecular data 110. In an embodiment, the pre-processing module 114 may be used to perform various types of processing, for example, as described further below with respect to FIGS. 2, 3A, 3B and 4, to the data from the one or more databases 104. The classifier 102, data 104, and pre-processing module 114 may be implemented and stored on a computer system such as the exemplary computer system described below with respect to FIG. 5. In an embodiment, imaging data 106, clinical data 108 and molecular data 110 may be associated with and stored on a hospital network. In one embodiment, classifier 102 is configured to use the imaging data 106, clinical data 108 and molecular data 110 to derive a probability of cancer for each pixel or voxel (or various groups of pixels or voxels) of an image within a user-defined time interval. The imaging data 106 may include a plurality of sets of imaging exams (e.g., 2D mammograms, 3D MRIs, 3D CTs). Each set of images is associated with a patient and includes two or more images that were sequentially acquired using a medical imaging device (e.g., mammography system, MRI system, CT system, etc.). In an embodiment, each image in a set of images has been acquired at a different point in time and each of the acquired serial images may be separated by, for example, days, weeks, months, years, etc. In one embodiment, the images in imaging data 106 are encoded using DICOM format. The clinical data 108 may include information about the patient associated with a set of images (or imaging exams) such as, for example, patient age, race/ethnicity, other demographic information, personal and family history, cancer risk factors (e.g., EGFR mutation, current/former smoker), and outcome information (e.g., whether or not the patient was diagnosed with cancer in the tissue of interest). In an embodiment, the clinical data is encoded in a structured manner, for example, exported from the electronic health record as a comma separated value file. The molecular data 110, if available, may include, for example, data about the status or presence of a germline mutation in the patient (e.g., BRCA1/2 mutation status) and sequencing data. As mentioned above, germline mutation in a patient may be identified using molecular or genomic analysis such as whole genome sequencing (WGS) or whole exome sequencing (WES).

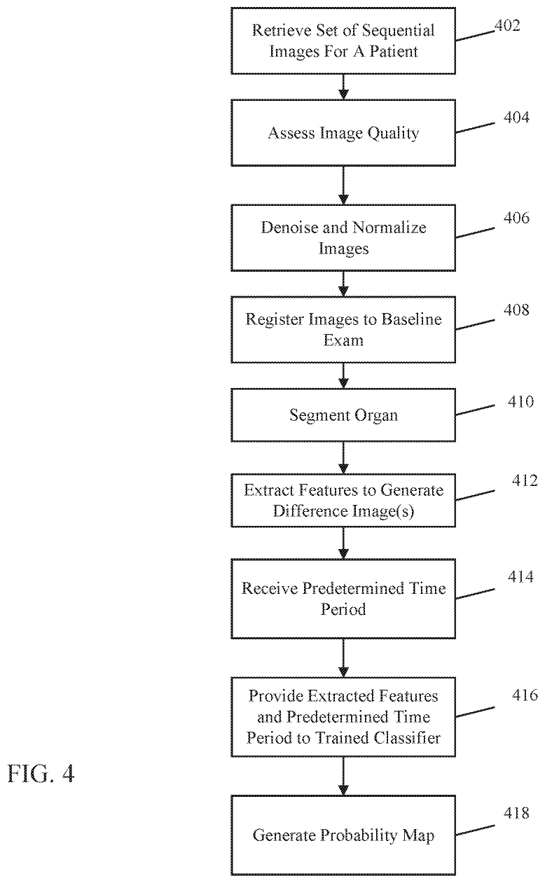

Pre-processing module 114 is configured to receive a selected set of images for a specific patient. The set of images for the patient includes at least two sequential images of a region (e.g., tissue(s) or whole organs) of interest associated with a selected cancer of interest. The pre-processing module 114 performs various processing steps on the set of images to generate difference image data (e.g., extracted features) that may be associated with or characterize changes in tissue characteristics as discussed further below with respect to FIG. 4. The difference image data is provided to the classifier 102. In an embodiment, clinical and molecular data associated with the patient may be included with the difference image data and also input into the classifier 102. A time interval i is selected (e.g., by a user) used to define a predetermined time period 116 for which the probability of cancer will be determined or computed, for example, the probability that a malignancy will appear in the pixel within i years. In an embodiment, classifier 102 may generate a probability map 112 of cancer formation at each pixel in a region of interest within time period t+i, where t is the current time and i is the selected time interval. In another embodiment, the probability of cancer may be determined for each voxel for the determined time period. The time interval i may span several magnitudes (e.g., days, weeks, months, etc.). The predetermined time period 116 is input into the classifier 102. For the selected patient case, classifier 102 uses the difference image data and predetermined time period inputs to assign a probability (a value between 0 and 1) to each pixel within an image region (e.g., for breast cancer pixels corresponding to the breast parenchyma), representing the likelihood that a malignancy will be found at the location of that pixel within the selected time interval (e.g., the user-defined time interval). The generated probability map 112 may be, for example, displayed on a display. In an embodiment, the generated probability map 112 may be overlaid on an image from the set of images for the patient. In an embodiment, the classifier 102 is a neural network (machine learning or deep learning) that has been trained for classifications and predictions for a particular type of cancer. As discussed further below, classifier 102 may be configured to provide quantitative predictions for the formation of a selected type of cancer such as, for example, breast cancer, prostate cancer, liver cancer, pancreas cancer, etc. The following description refers to embodiments and examples of the disclosed methods and system with respect to breast cancer, however, it should be understood that in other embodiments the methods and systems may be configured to provide quantitative predictions for other types of cancer.

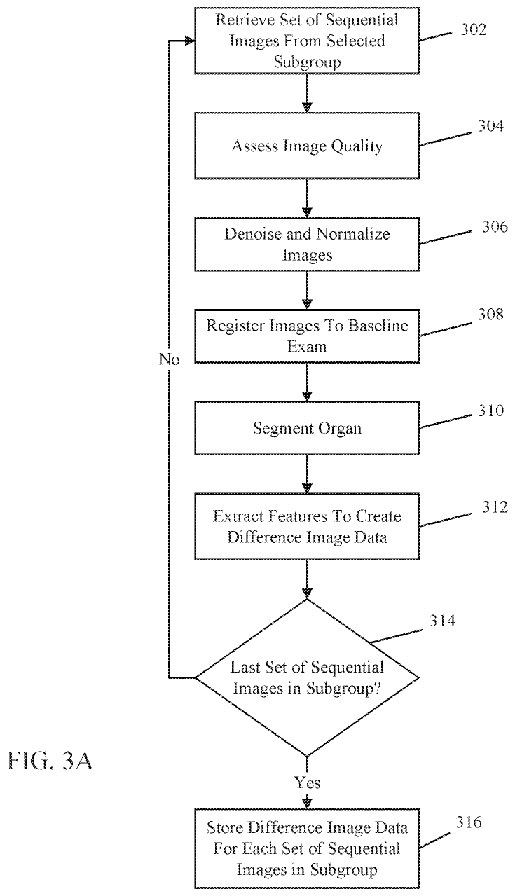

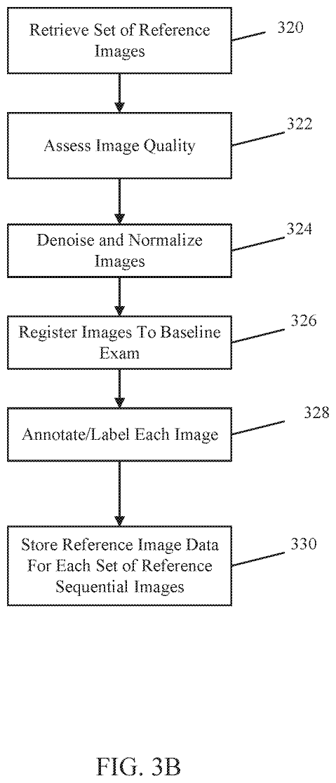

In an embodiment, classifier 102 is trained to classify on a pixel-level (for two dimensional images) or voxel level (for three dimensional images) the probability of cancer within time period t+i. FIG. 1B is a block diagram of a system for training a classifier of the system shown in FIG. 1A in accordance with an embodiment. As mentioned above, the imaging data 106 includes a plurality of sets of imaging exams (e.g., 2D mammograms, 3D MRIs, 3D CTs) and each set of imaging exams is associated with a patient and include two or more images that were sequentially acquired using a medical imaging device (e.g., mammography system, MRI system, CT system, etc.). For training of the classifier 102, pre-processing module 114 is configured to performs various processing steps on the plurality of sets of images to generate difference image data 120 (e.g., extracted features) as discussed further below with respect to FIGS. 2 and 3A. In addition, the pre-processing module 114 is configured to receive a selected set of reference images from imaging data 106 and perform processing steps on the set of reference images as discussed below with respect to FIG. 3B. In addition, the set of reference images is annotated by a human annotator (e.g., board-certified radiologist) to identify if a suspicious abnormality exists on each image in a set of reference images. The annotated reference data/labels 122 are provided as an input to the classifier 102 as well as the difference image data 120 to train the classifier for generating a prediction of the formation of cancer for a particular cancer type. As discussed further below with respect to FIG. 2, a different classifier may be trained for each of a number of different subgroups of the plurality of sets of images associated with the selected cancer type where each subgroup shares characteristics (e.g., based on demographic data, clinical data, molecular data, etc.).

FIG. 2 illustrates a method for training a classifier in accordance with an embodiment. At block 202, each set of images in the sequential imaging data 106 that is associated with a selected type of cancer (e.g., breast cancer) is identified. At block 204, the images within each identified set of images (or imaging studies) are organized sequentially to generate a set of sequential images. Each image, e.g., a mammogram, produced by a clinical device may be encoded using, for example, the DICOM format. The DICOM format is a standardized file format that includes both patient information in a structured header and the image data. From the DICOM header, the date of exam and image acquisition information such as device, exposure time, x-ray tube current, body part thickness, and energy (kVp) may be extracted. Additional information specific to the imaging modality is also available. For example, in mammograms, header information includes compression force and uniquely identifies breast images by laterality. This information may be combined to create a standardized, unique label for each imaging study (e.g., match images of the right breast, CC view).

At block 206, the sets of sequential images or exams are matched by modality, description, and relevant clinical and molecular data to generate a plurality of subgroups of the sets of sequential images. As mentioned above, in an embodiment individual prediction models (i.e., a classifier 102) may be generated based on each subgroups of cases that share similar characteristics. In one example for breast cancer, subgroups may be created for imaging modalities (e.g., 2D mammography versus 3D mammography), patients (e.g., younger patients <60 years old versus older patients ≥60 years old who tend to have less dense breasts), and clinical/molecular information, whenever available, such as race/ethnicity and mutation status (e.g., BRCA1/2). The purpose of creating a classifier for each subgroup is to reduce the variability that exists among different modalities, patients, clinical data and molecular data. Individual classifiers or models are trained on data from each subgroup (e.g., a classifier for younger women who are BRCA1/2 positive heterogeneous or extremely dense breasts). The images or exams may also be matched by laterality and other positioning information to ensure a consistent field of view. At block 208, a set of sequential images associated with a selected subgroup are identified for use in training a classifier for the subgroup. The training process described in references to blocks 208-216 below may be repeated for each subgroup to create a classifier for that subgroup.

At block 210, difference image data is generated for each set of sequential images in the subgroup. FIG. 3A illustrates a method for generating difference image data for training a classifier in accordance with an embodiment. At block 302, a set of sequential images from the selected subgroup is retrieved. At block 304, image quality is assessed. To ensure that identified differences in serial images reflect biological changes, a series of processing steps are performed to ensure that the image intensity values and field of view (i.e., visualized region of the breast) are normalized across all scans. First, the each image in the selected set of sequential images is classified as to whether the image is of sufficient quality to perform the analysis. In one embodiment, this step may be performed by initial intensity-based thresholding to identify regions where artifacts (e.g., caused by clips) or distortion exist. If a certain percentage of pixels in the image exceed the predefined threshold (e.g., 5% of pixels), the image is assessed as too poor quality to process, and the image will be not be processed further or used in training the classifier. In addition, anatomical landmarks that are observable in images may be detected and utilized to ensure consistent field of view and patient positioning. The anatomical landmarks may be detected using known methods.

At block 306, each image in the selected set of images (e.g., pixel intensity values for each image) are denoised and normalized. If an image is of sufficient quality, a denoising algorithm is applied to reduce acquisition-specific noise and enhance tissue contrast in a region of interest (e.g., the parenchymal region). In various embodiments, known denoising algorithms may be used. In one embodiment for breast imaging utilizing mammograms, a convolutional neural network that consists of 10 layers, 5 convolutional (encoder) and 5 deconvolutional (decoder) layers symmetrically arranged may be used for denoising and normalizing. Each layer is followed by a rectified linear unit (ReLU). The convolution neural network (CNN) with perceptual loss is trained to map between mammograms acquired at different compression force and tube current to a standardized value, essentially denoising and normalizing these images. Given that actual patient images at different tube currents and compressions are impractical to obtain, the network is trained using a physics-based simulation to generate multiple possible views of breast parenchyma under different acquisition parameters. Using the trained denoising CNN, mammograms that are acquired serially but perhaps with slight variations in acquisition settings are inputted into the model, and a normalized, denoised image is generated as the output. At block 308, a baseline image is selected from the set of sequential images and the remaining sequential images in the set are registered to the baseline image (or exam). In an embodiment, the selection of a baseline image is arbitrary. In another embodiment, the baseline image is the earliest acquired image in the set of sequential images. The images may be registered to the baseline image using known methods.

At block 310, segmentation of an organ or tissue of interest (or region of interest) is performed on each image in the set of sequential images. The organ or tissue of interest is based on the selected cancer type. Known methods for image segmentation may be used to segment the organ or tissue of interest. In an embodiment for breast imaging, an automated segmentation approach utilizing adaptive thresholding to delineate the breast parenchyma and an iterative “cliff detection” approach to delineate the pectoral margin, separating the breast tissue from pectoral muscle may be applied. Once segmented, the breast parenchyma region (foreground) may be resized to fit an image of fixed size (e.g., 1200×1200) and the background region is set to 0.

At block 312, an extraction process is performed on the segmented organ or tissue of interest (or region of interest) for each image to extract features per pixel in the segmented region and to generate difference image data, for example, a difference image for each pair of sequential images in the set of sequential images. For example, if there are three sequential images (image 1, image 2 and image 3) in the set of sequential images, two difference images are generated. One difference image between image 1 and image 2 and one difference image between image 2 and image 3. In an embodiment, the feature for each pixel is a quantitative representation of at least one underlying tissue characteristic. For example, as mentioned above, salient imaging features (e.g., intensity profile, shape, texture) may be quantitatively analyzed and associated with changes in tissue characteristics (e.g., microstructure, metabolic status, physiologic status, cytoarchitecture, etc.). The purpose of the extraction process is to generate features that best characterize observed differences between two sequential imaging scans in the set of sequential images. In an embodiment, rather than operate on the original (raw) image, the image may first be transformed into a different space that would help amplify features that have changed between the two serial scans. Various transformations may be used for this purpose. In an embodiment for breast imaging, the Phase Stretch Transform may be applied because of an interest in detecting textual differences in the breast parenchyma (e.g., structural alterations in breast tissue that may indicate environmental changes). In this embodiment, the input image is first transformed into the frequency domain using 2D or 3D fast Fourier transform, depending on imaging modality. A warped phase stretch transform is then applied on the image in this domain. The phase of the output image is then thresholded and postprocessed using morphological operators to enhance edge information within the image. Each image is transformed in the same manner. Taking two sequential transformed images, the difference between the two images is calculated and, for example, a difference image is generated. At block 314, it is determined whether the current set of sequential images being processed is the last set of sequential images in the subgroup. If the current set of sequential images being processed is not the last set of sequential images in the subgroup, the process returns to block 302 and another set of sequential images is selected and processed as described with respect to blocks 304-312. If the current set of sequential images being processed is the last set of sequential images in the subgroup, the difference image data (e.g., difference images) for each image in the set of sequential images is stored, for example, in the sequential image database 106 (shown in FIGS. 1A and 1B).

Returning to FIG. 2, at block 212, a plurality of reference images are selected from the plurality of sets of sequential images associated with the selected subgroup. In an embodiment, the set of reference images may be collected and annotated. The set of reference images is comprised of images with known cancer and non-cancer case outcomes that are labeled using available diagnostic information. In an embodiment, information from radiologists and pathologists may be used to determine where an area of suspicion exists in each reference image, and after biopsy, whether that region of suspicion is malignant (e.g., invasive ductal carcinoma). At block 214, reference data for training the classifier is generated for each reference image in the set of reference images. FIG. 3B illustrates a method for generating reference data for training a classifier in accordance with an embodiment. At block 320, the set of reference images is retrieved. At block 322, image quality is assessed for each reference image in a similar manner as described above with respect to block 304 in FIG. 3A. If a reference image is assessed as too poor quality to process, the image will be not be processed further or used in training the classifier. At block 324, each reference image (e.g., pixel intensity values for each image) is denoised and normalized in a similar manner as described above with respect to block 306 of FIG. 3A. At block 326, a baseline image is selected and each reference image in the set of reference images is registered to the baseline image (or exam). In an embodiment, the selection of a baseline image is arbitrary. At block 328, each reference image is annotated manually. For each reference image, a human annotator (e.g., board-certified radiologist) determines whether any suspicious areas exist. If a suspicious area is identified, the annotator outlines the region in which the suspicious abnormality exists on the image. In an embodiment, the outlines need only roughly correspond to regions of abnormalities. For example, regions of the most suspicious microcalcifications may be outlined. To account for potential variability in outlined regions, multiple versions of the region may be generated by randomly varying the boundaries. Positive examples are then generated by extracting patches from within the outlined regions. Positive examples are regions (e.g., two or more pixels) in a reference image that include areas the annotator identified as having a suspicious abnormality. Negative examples are generated by extracting patches that are outside the outlined regions but wholly within the region of interest (e.g. the tissue or organ of interest). Negative examples are regions in a reference image that do not include areas the annotator identified as having a suspicious abnormality. At block 330, the reference data for each reference image (e.g., the annotated reference image, the positive and negative examples, labels, etc.) is stored, for example, in the sequential image database 106 (shown in FIGS. 1A and 1B).

Returning to FIG. 2, the difference image data (block 210) and the reference data (block 214) are provided to the classifier for training. In one embodiment, individual pixels of each difference image is input into the classifier. In another embodiment, regions of each difference image may be input into the classifier. In yet another embodiment, features from the difference images (e.g., texture) may be input into the classifier. Additional data such as, for example, outcome information and follow up times (e.g., the time between when an image scan was performed and when a cancer outcome was determined) may also be provided to the classifier for training. At block 216, the classifier is trained. Example technologies for learning the temporal predictive model include, but are not limited to, convolutional neural networks (CNNs) conditioned on genetic abnormalities, possibly as an input to the network, that are trained to predict pixel- or voxel-level cancer probability given the time interval, i. In one embodiment, using the difference image data that is generated at block 210 and the reference data that is generated at block 214, a machine learning-based classifier is trained to generate a probability that the input pixel (or set of pixels) will represent cancer within a time interval i. In an embodiment, a feed-forward neural network may be used that is trained using a large set of positive and negative training patches generated as described above with respect to block 328 of FIG. 3B. The output layer of the network is a Cox regression. The hazard function is defined by:

h(i|Wₓ_{ₘ})=h₀(i)exp θₘ

where h(i|Wₓ_{ᵢ}) represents the hazard function within time interval i for patient case m with weighted features Wₓ_{ₘ}·h₀(i) is the baseline hazard. The variable θₘ is defined as the log hazard ratio for case m:

θₘ=G(Wₓ_{ₘ}+b)^{T}β

where G is the activation function, W defines the coefficient weight matrix between the input and hidden layers, and b is the bias term for each hidden node. The probability of the event to be observed occurring with case m at time i is given as:

l(β)=Σ_{C(m)=1}(θₘ−log Σᵢ_{ₙ}_{≥i}_{ₘ} exp(θₙ))

where l(β) is the partial log likelihood, C(m)=1 indicates the occurrence of a malignancy in a case, and n represents the set of cases where malignancy has not occurred before time interval i. The output of the model is the predicted probability of an event (diagnosis of malignancy) assigned to each pixel (or set of pixels) in the image.

Once a classifier has been trained, the classifier may be applied to a set of sequential images for a particular patient to generate a probability map. FIG. 4 illustrates a method for tissue classification in accordance with an embodiment. At block 402, a set of sequential images for a particular patient are retrieved, for example, from the sequential imaging data 106 (shown in FIG. 1A) and information about the patient case is determined, for example, demographics, mutation status, etc. The information about the patient case may be determined from, for example, the clinical data 108 (shown in FIG. 1A) and molecular data 110 (shown in FIG. 1A) in the databases 104. In one embodiment, the set of sequential images for the patient has not been previously processed by the tissue classification system (e.g., system 100 shown in FIG. 1A) to determine a probability of cancer formation. In another embodiment, a new sequential image has been acquired for the patient associated with a set of sequential images that includes prior images that have previously been processed by the tissue classification system (e.g., system 100 shown in FIG. 1A) to determine a probability of cancer formation. Preprocessing steps similar to those described above with respect to blocks 304-310 of FIG. 3A are performed at blocks 404-410 of FIG. 4 for each image in the set of sequential images for the patient. At block 412, an extraction process similar to that described above with respect to block 312 in FIG. 3A is performed on the segmented organ or tissue of interest (or region of interest) for each image to extract features per pixel in the segmented region and to generate difference image data, for example, a difference image for each pair of sequential images in the set of sequential images for the patient. At block 414, a predetermined time period for determining the probability of cancer formation is received, for example, from a user via a user input. At block 416, the extracted features (e.g., the difference image(s)) determined at block 412 and the predetermined time period received at block 414 is provided to a trained classifier (e.g., classifier 102 shown in FIG. 1A). At block 418, the classifier generates a probability map for the formation of cancer for the entire region of interest in a predetermined time period based on changes to the tissue detected in the difference image(s). The generated probability map may be, for example, displayed on a display at block 420. In one embodiment, the probability map may be overlaid on one of the sequential images from the set of sequential images for the patient (e.g., the most recent sequential image) and displayed on a display.

FIG. 5 is a block diagram of an example computer system that can implement the systems and methods described herein in accordance with an embodiment. The computer system 500 generally includes an input 502, at least one hardware processor 504, a memory 506, and an output 508. Thus, the computer system 500 is generally implementer with a hardware processor 504 and a memory 506. In come embodiments, the computer system 500 may be a workstation, a notebook computer, a tablet device, a mobile device, a multimedia device, a network server, a mainframe, one or more controller, one or more microcontrollers, or any other general-purpose or application-specific computing device.

The computer system 500 may operate autonomously or semi-autonomously, or may read executable software instructions from memory 506 or a computer-readable medium (e.g., hard drive a CD-RIOM, flash memory), or may receive instructions via the input from a user, or any other source logically connected to a computer or device, such as another networked computer or server. Thus, in come embodiments, the computer system 500 can also include any suitable device for reading computer-readable storage media. In general, the computer system 500 may be programmed or otherwise configured to implement the methods and algorithms described in the present disclosure.

The input 502 may take any suitable shape or form, as desired, for operation of the computer system 500, including the ability for selecting, entering, or otherwise specifying parameters consistent with performing tasks, processing data, or operating the computer system 500. In some aspects, the input 502 may be configured to receive data, such as imaging data, clinical data or molecular data. In addition, the input 502 may also be configured to receive any other data or information considered useful for implementing the methods described above. Among the processing tasks for operating the computer system 500, the one or more hardware processors 504 may also be configured to carry out any number of post-processing steps on data received by way of the input 502.

The memory 506 may contain software 510 and data 512, such as imaging data, clinical data and molecular data, and may be configured for storage and retrieval of processed information, instructions, and data to be processed by the one or more hardware processors 504. In some aspects, the software 510 may contain instructions directed to implementing one or more machine learning algorithms with a hardware processor 504 and memory 506. In addition, the output 508 may take any form, as desired, and may be configured for displaying images, patient information, probability maps, and reports, in addition to other desired information. Computer system 500 may also be coupled to a network 514 using a communication link 516. The communication link 516 may be a wireless connection, cable connection, or any other means capable of allowing communication to occur between computer system 500 and network 514.

Computer-executable instructions for tissue classification according to the above-described systems and methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present invention has been described in terms of one or more preferred embodiments, and it should be appreciated that many equivalents, alternatives, variations, and modifications, aside from those expressly states, are possible and within the scope of the invention.

1. A method for tissue classification, the method comprising:
receiving at least two images associated with a patient, the at least two images being of a tissue;
identifying a region of interest in the at least two images;
analyzing the region of interest to identify changes in the tissue;
generating a probability map of the region of interest based on the changes in the tissue, the probability map indicating a likelihood of formation of cancer in the tissue within a predetermined time period; and
displaying the probability map on a display.

2. The method according to claim 1, wherein analyzing the region of interest to identify changes in the tissue includes identifying changes in the tissue based on a comparison of one of the at least two images to at least one previously acquired image in the at least two images.

3. The method according to claim 1, wherein the at least two images are two-dimensional images and the region of interest includes a plurality of pixels and generating a probability map comprises generating a probability of cancer formation for each pixel in the region of interest.

4. The method according to claim 1, wherein the at least two images are three dimensional images and the region of interest includes a plurality of voxels and generating a probability map comprises generating a probability of cancer formation for each voxel in the region of interest.

5. The method according to claim 1, wherein analyzing the region of interest to identify changes in the tissue comprises:
applying a denoising process to the at least two images;
normalizing the at least two images;
segmenting the region of interest; and
extracting a feature for each pixel in the region of interest.

6. The method according to claim 5, wherein the feature for each pixel is a quantitative representation of at least one underlying tissue characteristic.

7. The method according to claim 5, wherein extracting a feature for each pixel in the region of interest comprises generating a difference image between two sequential images in the at least two images.

8. The method according to claim 1, wherein the at least two images have an associated set of clinical data for the patient

9. The method according to claim 1, wherein the at least two images have an associated set of molecular data for the patient.

10. The method according to claim 5, further comprising:
selecting a baseline image from the at least two images; and
registering at least one image in the at least two images to the baseline image.

11. A system for tissue classification comprising:
at least one database;
a pre-processing module coupled to the at least one database and configured to receive at least two images associated with a patient, the at least two images being of a tissue, to identify a region of interest in the at least two images, and to analyze the region of interest to identify changes in the tissue; and
a classifier coupled to the at least one database and the pre-processing module, the classifier configured to generate a probability map of the region of interest based on the changes in the tissue, the probability map indicating a likelihood of formation of cancer in the tissue within a predetermined time period.

12. The system according to claim 11, wherein the at least two images are two-dimensional images and the region of interest includes a plurality of pixels and the classifier is configured to generate a probability map by generating a probability of cancer formation for each pixel in the region of interest.

13. The system according to claim 11, wherein the at least two images are three dimensional images and the region of interest includes a plurality of voxels and the classifier is configured to generate a probability map by generating a probability of cancer formation for each voxel in the region of interest.

14. The system according to claim 11, wherein the changes in the tissue are changes in at least one quantitative representation of an underlying tissue characteristic.

15. The system according to claim 11, wherein analyzing the region of interest to identify changes in the tissue includes identifying changes in the tissue based on a comparison of one of the at least two images to at least one previously acquired image in the at least two images.

16. The system according to claim 11, wherein the at least one database includes sequential imaging data, clinical data, and molecular data.

17. The system according to claim 11, wherein the classifier is a neural network.

18. The system according to claim 11, wherein the classifier is associated with a type of cancer and the probability map indicates a likelihood of formation of the type of cancer associated with the classifier.
